(19) United States
(12) Patent Application Publication
(10) Pub. No.: US 2016/0303541 A1
(43) Pub. Date: Oct. 20, 2016

(54) METHOD OF PREPARING CHROMATOGRAPHIC MATERIALS

(71) Applicant: NEW PROTEINTECH INC., Haifa Nesher (IL)

(72) Inventor: Karina ROZHETSKY, Haifa (IL)

(73) Assignee: New Proteintech, Inc., Newark, DE (US)

(21) Appl. No.: 15/037,054

(22) PCT Filed: Nov. 17, 2014
(86) PCT No.: PCT/IL2014/050995

§ 371 Date: May 17, 2016

Related U.S. Application Data
(60) Provisional application No. 61/905,238, filed on Nov. 17, 2013.
(60) Provisional application No. 61/905,239, filed on Nov. 17, 2013.

(51) Int. Cl. B01J 20/26; B01D 15/36; B01J 20/28; B01D 15/42; C07K 1/16; B01D 15/38; B01D 15/32

(52) U.S. Cl. CPC B01J 20/267; B01D 15/3847; B01D 15/362; B01D 15/327; B01D 15/424; C07K 1/16; B01J 20/28004; B01J 20/28085

(57) ABSTRACT

A sorbent material comprises a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers. The sorbent is used in chromatographic columns to promote binding of Immunoglobulin G (IgG) from blood plasma, for its isolation, such that the isolated IgG is then extracted from the sorbent. The sorbent is also used in chromatographic columns to promote binding of Immunoglobulin G (IgG) monoclonal antibodies from transgenic milk, for their isolation, such that the isolated IgG monoclonal antibodies are then extracted from the sorbent.

### CROSS REFERENCES TO RELATED APPLICATIONS

This application is related to and claims priority from commonly owned US Provisional patent applications: Ser. No. 61/905,238, entitled: Method of Preparing New Chromatographic Materials and New Chromatographic Materials for Highly Selective Chromatography, filed Nov. 17, 2013, and, Ser. No. 61/905,239, entitled: Method of Preparing Mixed Mode Resins and Mixed Mode Resins for Highly Selective Chromatography, filed Nov. 17, 2013, the disclosures of which are incorporated by reference in their entirety herein.

### TECHNICAL FIELD

The present invention relates to preparing new resins for highly selective chromatography applications.

### BACKGROUND

As the worldwide demand for blood-based products continues to increase, effective and economic methods for biotechnological purified products are sought. Included in these many methods is the isolation of numerous proteins, enzymes, hormones and other bio-active compounds from various physiological fluids, nature sources, and cultural media. Considerable attention has been given to isolation and purification technologies related to the field of chromatography.

Conventional chromatographic materials known in the art do not lend themselves sufficiently and effectively to many vitally important purification applications. Purified products are used in therapeutics, cosmetics and foods, for example. Among the shortcomings of applying conventional chromatographic materials are: multistep purification schemes having pretreatment operations; large losses of material during purification; and a resultant low yield and high cost of the purified product.

Another problem involves isolation and purification of Immunoglobulin G (IgG) from raw human blood plasma as start source. Current IgG purification technologies use fraction II+III paste of Cohn process. However, during the stages of the aforementioned purification, at least 30% of the IgG is lost during the numerous manufacturing steps. As a result, commercial yield can, therefore, be as low as 50% of the original IgG.

### SUMMARY

Embodiments of the present invention are directed to a sorbent material, which comprises a plurality of cross linked monovinyl monomers defining a matrix. The monovinyl monomers are in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers. The sorbent is used in chromatographic columns to promote binding of Immunoglobulin G (IgG) from blood plasma, for its isolation, such that the isolated IgG is then extracted from the sorbent. The sorbent is also used in chromatographic columns to promote binding of Immunoglobulin G (IgG) monoclonal antibodies from transgenic milk, for their isolation, such that the isolated IgG monoclonal antibodies are then extracted from the sorbent.

Embodiments of the present invention are based on new highly selective chromatographic materials.

Embodiments of the present invention comprise chromatographic materials and their applications. Among the materials are bi-functional Mixed Modes chromatographic materials including weak cation exchanging functional groups and hydrophobic functional groups, new mono-functional anion exchange resins and new materials of hydrophobic interaction chromatography. The chromatographic materials are directed to improving the chromatographic purification processes of protein, enzyme, glycoprotein, polysaccharide, inter alia, from various raw sources.

Embodiments of the present invention are directed to improve the efficacy of technology for isolation and purification of IgG from raw human plasma for therapeutic purposes.

Embodiments of the present invention include a method of IgG purification from raw human blood plasma. The method comprises chromatographic purification of IgG from raw human blood plasma by using the chromatographic structures of the present invention.

The present invention in some embodiments includes chromatographic structures and method for their preparation. The chromatographic structures combine material properties, including a principle of ion exchange, hydrophobic, and the required pore size. These chromatographic materials provide the present invention with advantages over various chromatographic methods for antibody manufacturing. Advantages include: avoiding of preliminary treatment; substituting a centrifugation step (or microfiltration); demonstration of highly selective sorption from raw sources; full sorption recovery; high hydrodynamic properties; stability of structure volume; and flow rate to 600 ml/hr.

The structure of the materials of the present invention is applicable for low pressure liquid chromatography (LPLC), and withstands pressures of at least 3 bar. The bi-functional Mixed Mode material or media of the present invention can be integrated as first general step for capturing of IgG (and numerous other proteins) from various raw sources.

In contrast to other conventional resins, the Mixed Mode (e.g., sorption material or media) of the present invention, provides approximately double the antibody binding capacity. IgG required sorption conditions in the pH range of 6 to 7 serve to provide high IgG purity using Mixed Mode. The present invention is such that a new hydrophobic interaction chromatography (HIC) with required pore sizes are used for polishing (IgG finished purification). A method of IgG purification using structures of embodiments of the current invention consists of 2 to 3 steps.

The present invention eliminates the need for centrifuges and desalination, as high ion exchange capacity, hydrophobic/hydrophilic balance and pore sixes, e.g., large pore sizes, in the sorbent material of the present invention includes active elements necessary to isolate the IgG and IgG monoclonal antibodies.

The present invention is able to isolate the IgG from blood plasma, by utilizing a polymeric matrix, with parameters adjustable to optimize ion exchange, balance hydrophobicity and hydrophilicity, as well as utilize mimetic properties through granule particle sizes and associated porosities, of interconnected pores.

The present invention avoids many preliminary preparation steps to purify plasma or transgenic milk, as the present invention avoids preliminary treatments; substitutes a centrifugation step (or microfiltration); demonstrates highly selective sorption from raw sources; provides full sorption recovery; exhibits high hydrodynamic properties; shows stability of structure volume; and exhibits a flow rate to 600 ml/hr. The structure is applicable for low pressure liquid chromatography (LPLC) and withstands pressures of at least 3 bar. The mixed mode (e.g., sorption material) of the present invention can be integrated as first general step for capturing of IgG (and numerous other proteins) from various raw sources.

Embodiments of the present invention include a method of human IgG purification from transgenic animal milk. The method comprises chromatographic purification of human IgG, e.g., IgG monoclonal antibodies, from transgenic milk by using the chromatographic structures and sorbent materials of the present invention.

Embodiments of the present invention are directed to a sorbent material (or media). The sorbent material comprises: a plurality of cross linked monovinyl monomers (e.g., co-monomers) defining a matrix, in a ratio of the volume of hydrophobic monovinyl monomers (e.g., co-monomers) to hydrophilic monovinyl monomers (e.g., co-monomers) of approximately 5:95 to approximately 40:60, the matrix being bufferable to pH ranges from approximately 5 to approximately 9, and, particles of particle sizes between approximately 10 micrometers to approximately 300 micrometers.

Optionally, the particle sizes are between approximately 100 micrometers to approximately 200 micrometers.

Optionally, the particles include pores of sizes of approximately 500 angstroms to approximately 3 micrometers.

Optionally, the pores are interconnected to each other.

Optionally, the volume of hydrophobic monovinyl monomers to hydrophilic monovinyl monomers is approximately 20:80.

Optionally, the matrix is bufferable to a pH of approximately 7.

Optionally, the monovinyl monomers include carboxyl groups from approximately 0.1 meq/ml to approximately 1.5 meq/ml.

Optionally, the carboxyl groups are approximately 0.7 meq/ml.

Optionally, the monovinyl monomers include one or more of: monovinyl acids including acrylic acids and hydrophobic vinyl-containing compounds, including butylacrylate, tert-butylacrylate, butyl methacrylate, octyl and phenyl acrylate and methacrylates.

Optionally, the monovinyl monomers are include one or more of: vinyl contained compounds, including substituted amines, including ethyleneically substituted amines, allylamines, N-alkylethylenamine and dimethylaminoethyl methacrylate.

Optionally, the monovinyl monomers include one or more of: hydrophobic vinyl contained compounds, including butylacrylate, tert-butylacrylate, butyl methacrylate, octyl and phenyl acrylate and methacrylates.

Other embodiments of the present invention are directed to a method for isolation of Immunoglobulin G (IgG) from blood plasma. The method comprises obtaining (or alternately providing) a sorbent material comprising: a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers. The sorbent material is placed into a chromatographic column; and, blood plasma is passed through the chromatographic column to isolate the IgG.

Optionally, the method additionally comprises: separating the isolated IgG of the blood plasma from the sorbent material.

Optionally, the separation of the IgG of the blood plasma from the sorbent material is performed by elution.

Optionally, the blood plasma is raw blood plasma.

Optionally, the raw blood plasma is mammalian blood plasma.

Optionally, the raw blood plasma is human blood plasma.

Another embodiment of the present invention is directed to a method for isolation of Immunoglobulin G (IgG) monoclonal antibodies from whey of milk The method comprises: obtaining (or alternately, providing) a sorbent material comprising: a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers. The sorbent material is placed into a chromatographic column, and, the whey protein of the milk is passed through the chromatographic column to isolate the IgG monoclonal antibodies.

Optionally, the milk includes transgenic milk.

Optionally, the transgenic milk is mammalian.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow diagram of processes in accordance with the present invention;

FIG. 2 is purification of immunoglobulin G (IgG) from raw undiluted human blood plasma on the bi-functional Mixed mode in accordance with embodiments of the present invention;

FIG. 3 is purification of immunoglobulin G (IgG) from raw human blood plasma by anion exchangers in, in accordance with embodiments of the present invention;

FIG. 4 is purification of immunoglobulin G (IgG) from human blood plasma in accordance with embodiments of the present invention;

FIG. 5 is a flow diagram of another processes in accordance with the present invention;

FIG. 6 is a diagram of sorption test of whey proteins on the sorbent material in a chromatographic column in accordance with embodiments of the present invention;

FIG. 7 is diagram of the chromatography of whey proteins in accordance with embodiments of the current invention;

FIG. 8 is diagram of the chromatography of whey proteins by large pore size at an initial pH of whey, in accordance with embodiments of the present invention; and,

FIG. 9 is diagram of the chromatography of whey proteins on the small pore size chromatographic column in accordance with embodiments of the present invention.

### DETAILED DESCRIPTION

I. Purification of Immunoglobulin G (IgG) from Raw Plasma

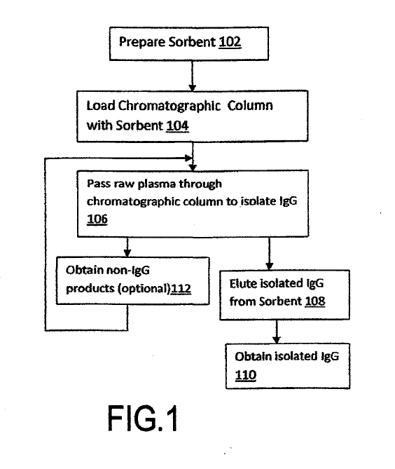

Attention is initially directed to FIG. 1, a flow diagram detailing an exemplary process and/or processes for purifying IgG from plasma, for example, raw human plasma, from human blood. The blocks of the flow diagram are also referenced throughout this section.

Initially, a sorbent is prepared at block 102. The sorbent is then loaded into a chromatographic column at block 104. The process moves to block 106, where the plasma, for example, raw human plasma, is passed through the sorbent loaded chromatographic column to isolate the IgG, at block 106. The isolated IgG is bound to the sorbent.

The process moves from block 106 to block 108, where the isolated IgG is eluted from the sorbent, in order obtain the isolated the IgG, at block 110. This isolated IgG is now suitable for numerous products.

Returning to block 106, the non-IgG products, which did not bind to the sorbent of the chromatographic column may optionally be collected. These products may again be passed through the same sorbent-loaded chromatographic column, or a different, but similar sorbent-loaded chromatographic column, for example, including the sorbent of the present invention, as the process returns to block 106, from where it resumes.

The processes of blocks 102, 104, 106, 108, 110, and 112 may be repeated for as long as desired.

### A. Prepare Sorbent (Mixed Mode Preparation)(FIG. 1, Block 102)

Methods for manufacturing structures, for example, matrices of polymeric monomers, for example, which are cross linked and formed of both hydrophobic monomers (e.g., co-monomers) and hydrophilic monomers (e.g., co-monomers), are described herein below, including methods comprising a suspension polymerization by principle of free radical copolymerization consisting pre-polymerization stage

One embodiment of present invention comprises co-monomers, preferably monovinyl monomers selected from a group consisting of any monovinyl weak acid, including, for example, acrylic acids and hydrophobic vinyl-containing compounds such as butylacrylate, tert-butylacrylate, butyl methacrylate, octyl and phenyl acrylate and methacrylates.

Alternately, the co-monomers are from the from a group including any weakly basic vinyl contained compounds such as substituted amines preferably ethyleneically substituted amines such as allylamines, N-alkylethylenamine, dimethylaminoethyl methacrylate.

Alternately, the monovinyl co-monomers are from a group including hydrophobic vinyl contained compounds such as butylacrylate, tert-butylacrylate, butyl methacrylate, octyl and phenyl acrylate and methacrylates. These materials are new resins of hydrophobic interaction chromatography (HIC).

For example, based on volume, the ratio of hydrophobic monovinyl monomers to hydrophilic monovinyl monomers is approximately 5:95 to approximately 40:60, and for example, approximately 20:80. The sorbent material, for example, is such that of the monovinyl monomers include carboxyl groups from approximately 0.1 meq/ml to approximately 1.5 meq/ml (milliequivalents/milliliter), for example, the carboxyl groups are approximately 0.7 meq/ml.

The cross-linking agents for the aforementioned monomers, e.g., co-monomers, are, for example, long chain cross-linking agents having at least two repeated carbon groups in the chain and preferably from 2 to 10 repeated carbon groups. The cross linking agent is a polyvinylmonomer selected from a group consisting of hexahydro-1, 3, 5-triacryloyltriazine (HTA), triallylisocyanurate (TAIL), p-phenylenedimethacrylamide (p-PHDMA), N,N′-methylenediacrylamide (MDAA), N,N′-ethylenedimethacrylamide (EDMA), N,N′-hexamethylenedimethacrylamide (HMDMA), pentaerythritol triacrylate (PETA), and pentaerythritol tetraacrylate (PETETA).

The chromatographic materials of the present invention are based on a free radical polymerization method. The initiator in the method is a free radical initiator selected from a group consisting of ammonium persulfate, a 1, 1′-azobis (cyclohexanecarbonitrile). Co-monomers and an initiator are incubated in a solution which is preferably selected from a group consisting of organic solvents, for example mixtures of organic solvents.

Additionally or optionally, co-monomers and an initiator are incubated in a solution which is preferably a mixture of polyethylene glycol and butyl alcohol, cyclohexanol and decyl alcohol, lauryl alcohol. Optionally, a co-polymerization solution comprises a mixture of acetic acid and polyethylene glycol, formamide, butyl alcohol, cyclohexanol and decyl alcohol. A pre-polymerization stage consists of dissolving co-monomers and initiators in a selected solvent.

An exemplary ratio of co-monomer mixture to selected solvent ranges from 1 to 4. An exemplary solvent for incubating of a pre-polymerization mixture consists of a mixture of some solvent components. All polymer structures are prepared by a suspension polymerization method with a prepolymerization step. Dispersion media includes an aqueous solution of inorganic salt. The ratio of dispersion medium to pre-polymerization phase is preferably from about 1 to 4 or about 1 to 5 volume/volume.

All polymer structures are prepared by suspension polymerization with a prepolymerization step. Monovinyl monomers, cross-linking agents and initiator are dissolved in the selected solution. During the pre-polymerization step free radical polymerization is produced at a temperature of 50 to 80° C. to achieve the desired viscosity. A viscous pre-polymerization dispersion phase is incorporated in a dispersion medium at a temperature of 70-80° C. and the resulting product is produced at 80-95° C. A dispersion medium is a 20% solution of sodium sulfate and/or optionally a water solution of sodium chloride.

The resulting product is prepared in terms of spherical granules (particles) ranging (in average particle size, e.g., diameter) from 10 μm-300 μm and, for example, approximately 100 μm to 200 μm. The granules (e.g., particles) include pores of sizes of approximately 500 angstroms to approximately 3 micrometers, with the pores interconnected to each other. The granules are prepared by washing by glacial acetic acid, water, 1N sodium hydroxide (NaOH), 1 M hydrochloride (HCl), and water. After washing, granules are wet fractionated.

Prepared sorbents are charged in a chromatographic column. The sorbent is equilibrated by a buffer solution 0.1M sodium acetate at pH of approximately 5 to approximately 9 and, for example, approximately pH 7 in the column. Plasma, for example, raw human plasma, from human blood, by initial pH ˜7.4 is loaded into the column. After washing of the column by an equilibrated buffer desorption of binding IgG is performed. Desorption is produced by step gradient with solution of 0.1 M citrate buffer by differing in pH: 5; 5.5; 6.0; in end 1 M NaOH.

Some examples are described to demonstrate possibilities of the structures of embodiments of the present invention. The resultant data is associated with the separation and purification of IgG from raw human plasma on the sorbent material or media of the present invention. High selective sorption results from raw start plasma on a first step and second step. Full purification (polishing) IgG is performed in a third step. Conditions of sorption-elution offer the prospect of IgG isolation with high purity and full recovery. This is due, for example, to factors and parameters, such as pH of sorption-elution, hydrophilic-hydrophobic balance of structure and size of pores.

The examples described below are non-limiting, and as such, do not limit the wide application possibilities of polymer chromatographic materials of embodiments of the present invention.

### A.1. Mixed Mode (Sorbent) Preparation

### Example

A 250 ml flask is provided with a stirrer, an inlet pipe for argon and a dropping funnel. Into this flask, 120 ml of 20% sodium sulfate is poured and argon is bubbled for 20 minutes. At the same time, 2 ml of phenyl methacrylate, 3 ml of methacrylic acid, 0.921 g of HTA, 2 ml of laurylic alcohol, 11 ml butanol, 11 ml of cylohexanol are added. After dissolution of the co-monomers, 0.06 g 1,1′-azobis(cyclohexanecarbonitrile) is added. Copolymerization is conducted at a temperature of 60° C. for 10 minutes to obtain a viscosity pre-polymer. Pre-polymer is dispersed into 120 ml of 20% sodium sulfate. The temperature is maintained at 60° to 70° C. for 30 minutes. The temperature is then increased to 100° C. and the reaction is continued at that temperature for 1 hour. Next after cooling to a temperature of 25° C., the resultant spherical granules are separated from the dispersion phase. Granules are then washed with glacial acetic acid, then with water, next with an aqueous solution of 1 M NAOH, then with 1M aqueous solution of hydrochloric acid and finally with water. The yield was 5.6 g. The prepared copolymer is fractionated in the wet state in fractions: 200 μm, 100 μm, and less then 100 μm.

These granules had pores of an average size of approximately 1000 Angstroms, with the pores being interconnected. The hydrophobic to hydrophilic volume ratio of co-monomers was approximately 20:80 and ionic concentrations for ion exchange was approximately 0.7 meq/ml (milliequivalents/milliliter) of carboxyl groups (from the methacrylic acid).

B. Chromatography of IgG from Raw Human Plasma

4 ml of Mixed Mode fraction 100 μm-200 μm granules (particles) is introduced (loaded) into the column (FIG. 1, block 104) having an inner diameter of 1.6 cm, a height of 2 cm, and is conditioned with 1M NaOH and 1M HCl and Distilled Water (DW). Finally, the sorbent is equilibrated with 0.1 M acetate buffer at pH 7 Sorption in the column: raw human plasma a flow rate of 0.6 ml/min (FIG. 1, block 106).

Washing: 0.1 M acetate buffer pH 7

Elution: 0.1 M citrate buffer differing in pH: 5; 5.5; 6.0; in end 1 M NaOH (FIG. 1, block 108).

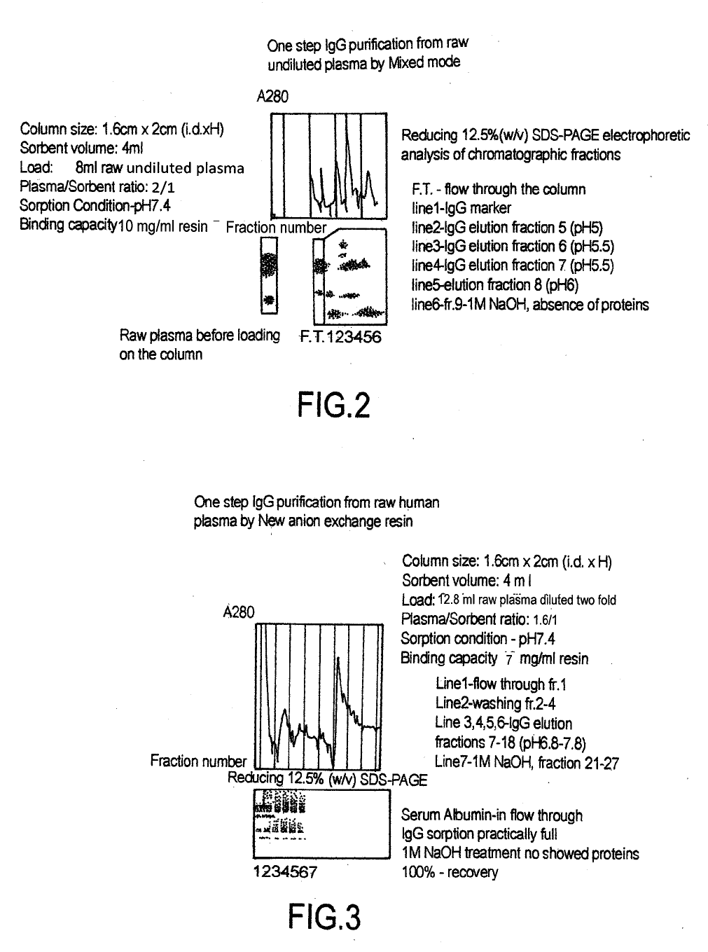

Results of chromatographic separations are shown in FIGS. 2-4. There are profiles of proteins eluted from column (graphs) and reducing 12.5% SDS-PAGE (polyacrylamide gel electrophoresis) analysis of the eluted peaks stained with Coomassie Brilliant Blue G-250.

FIG. 2 shows a one step purification of immunoglobulin G (IgG) from raw undiluted human blood plasma on the bi-functional structure based on weak acid and hydrophobic co-monomers presents by separation graph and reduced electrophoresis (PAGE). Elution fractions 5 of separation graph accords to the line 2 of electrophoresis image, elution fraction 6 accords to the line 3 of electrophoresis image, elution fraction 7 accords to the line 4 of electrophoresis, elution fraction 8 accords to the line 5 of electrophoresis. Electrophoresis analysis of elution fractions demonstrate high purity of isolated IgG without admixture of human serum albumin. Finished elution by 1M sodium hydroxide demonstrates absence of A 280 nm absorption, thus absent any proteins.

As shown in FIG. 3, a one-step purification of immunoglobulin G (IgG) from raw human blood plasma is performed using an anion exchange resin presents by separation graph and reduced electrophoresis (PAGE). Fraction 1-4 of flow-through and washing of graph accord to the line 1 and line 2 on the electrophoresis image. Full serum albumin is isolated in flow-through without IgG. Elution fraction 7-18 of graph according to the lines 3, 4, 5 6, of electrophoresis image. It is shown practical full sorption of IgG without human serum albumin admixture. Elution fractions 21-27 of separation graph according to the line 7 of the electrophoresis image of FIG. 3. Here, it is shown the absence of any proteins. High absorption A280 nm of elution fractions 21-27 causes due to dissolved a low molecular admixtures in 1 M sodium hydroxide.

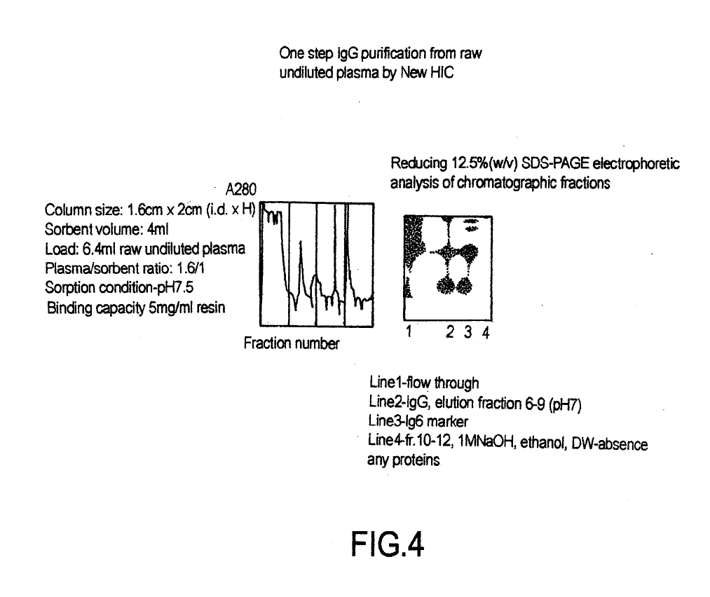

In FIG. 4, a one step purification of immunoglobulin G (IgG) from human blood plasma by HIC material employs a separation graph and reduced electrophoresis (PAGE). Fractions 1-3 of flow-through of graph accord to the line1 I of electrophoresis image. Elution fractions 6-8 of graph accord to the line 2 of electrophoresis image. It is shown high purity of isolated IgG without admixture of human serum albumin and others plasma proteins according to the marker line 3. Elution fractions 10-12 of separation graph accord to the line 4 of electrophoresis image. It is shown absence of any proteins. High absorption A280 nm of elution fractions 10-12 causes due to dissolved low molecular admixtures. PAGE results of all three figures demonstrate full sorption recovery because treatment with 1 M NaOH of chromatographic column after ending of sorption—the elution cycle did not show any proteins.

II. Purification of IgG from Transgenic Milk

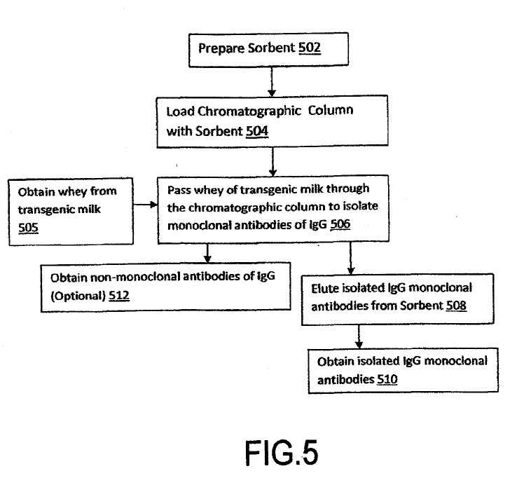

Attention is initially directed to FIG. 5, a flow diagram detailing an exemplary process and/or processes for purifying IgG monoclonal antibodies from transgenic milk, for example, the milk from a transgenic mammal. The blocks of the flow diagram are also referenced throughout this section.

Initially, a sorbent is prepared at block 502. The sorbent is then loaded into a chromatographic column at block 504. The process moves to block 506, where the whey protein, in liquid form, hereinafter “whey”, is passed through the sorbent loaded chromatographic column to isolate the IgG monoclonal antibodies, at block 506. The isolated IgG monoclonal antibodies are bound to the sorbent.

Prior to block 506, at block 505, the whey is obtained from the transgenic milk by conventional processes of obtaining whey from milk. For example, to produce whey, the transgenic milk is heated and rennet or an edible acid is added. This causes the transgenic milk to coagulate or curdle, separating the milk solids (curds) from the liquid whey.

The process moves from block 506 to block 508, where the isolated IgG monoclonal antibodies are eluted from the sorbent, in order obtain the isolated IgG monoclonal antibodies, at block 510. This isolated IgG is now suitable for numerous products.

Returning to block 506, the non-IgG monoclonal antibody products, which did not bind to the sorbent of the chromatographic column may optionally be collected, at block 512.

The processes of blocks 502, 504, 506, 508, 510, and 512 may be repeated for as long as desired.

### A. Prepare Sorbent (Mixed Mode Preparation)(FIG. 1, Block 502)

The Mixed Mode (sorbent)—i.e. a new resin combination and method for its preparation—serves to combine some important material properties, including a principle of ion exchange, hydrophobicity, and porosity.

Mixed Mode sorbents are charged in a chromatographic column. The sorbent is equilibrated by a buffer solution 0.1M sodium acetate at pH 6 in the column. Milk whey (milk is freed from casein) pH 4.5 treated to pH 7 (some drops of 1N NaOH) is loaded into the column. After washing of the column by an equilibrated buffer sorption of binding IgG is performed. Desorption is produced by step gradient with solution of 0.36 M ammonium acetate (NH4Ac) buffers by differing pH 5, pH 6, pH 7, and pH 8 in end 1N NaOH.

Some examples are described to demonstrate possibilities of the structures of embodiments of the present invention. The resultant data is associated with the separation and purification of whey proteins on the structures and media of the present invention. It is shown that all whey proteins in the number IgG are sorbed by the new structures. Conditions of sorption offer the prospect of isolation of proteins with high purity and full recovery are due, for example, to factors and parameters such as pH of sorption, hydrophobic contribution of structure and size of pores.

The examples described do not limit the wide application possibilities of polymer chromatographic materials of embodiments of the current invention.

The sorbent material is prepared in accordance with the sorbent material or media detailed above.

### A.1. Mixed Mode (Sorbent) Preparation

This sorbent preparation is in accordance with the EXAMPLE detailed above for “Mixed Mode Preparation.”

### B. Chromatography of Whey Proteins

4 ml of Mixed Mode fraction 100 μm-200 μm is introduced into the column having an inner diameter of 1.6 cm, a height of=2 cm, and is conditioned with 1M NAOH and 1M HCl. Finally, the sorbent is equilibrated with 0.1 M acetate buffer at pH 4.7 sorption in the column: 50 ml of the milk whey is applied at a flow rate of 0.6 ml/min.

Washing: 0.1 M acetate buffer pH 4.7.

Elution: 0.36 M ammonium acetate buffers differing in pH: 4.5; 5; 5.2; 5.5; 6.0; 7.0; 8.0 in 1 M NaOH.

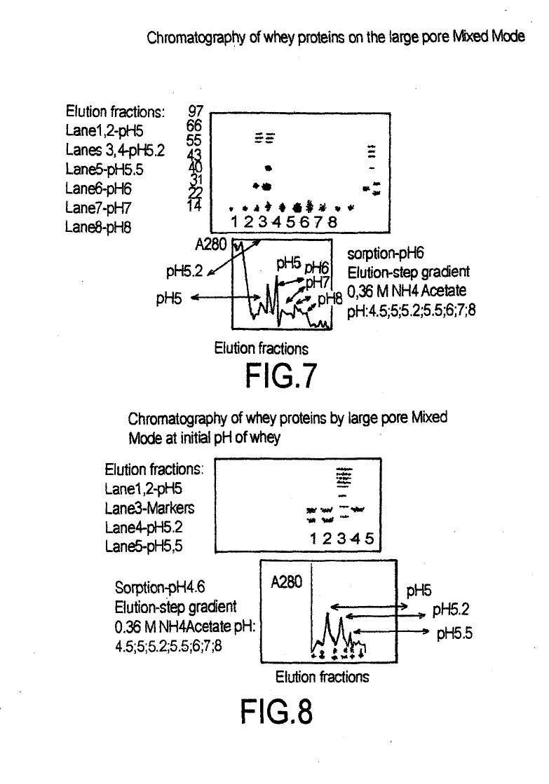

Results of chromatographic separations are shown in FIGS. 7, 8, and 9. There are profiles of whey proteins eluted from column (graphs) and reducing 12.5% SDS-PAGE (polyacrylamide gel electrophoresis) analysis of the eluted peaks stained with Coomassie Brilliant Blue G-250.

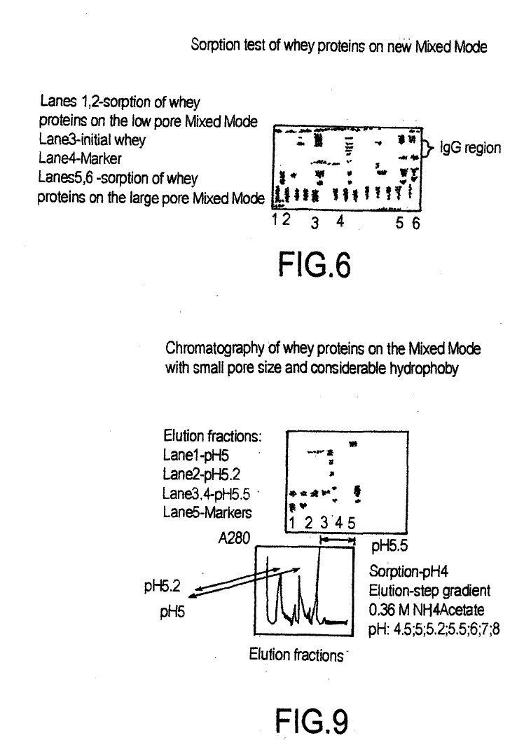

FIG. 6 shows a sorption test of whey proteins on the mixed mode resin. Line 1 represents proteins of raw initial whey.

Lines 2, 3 are fractions eluted with 0.36M NH4Ac buffer pH 5 to 5.5 from chromatography on the large pore Mixed Mode.

This obtained data demonstrates the sorption of all whey proteins on the large pore Mixed Mode (lanes 5, 6) according to the content of raw initial whey (line 3). IgG sorption is shown. Low molecular mass whey proteins are separated on the small pore new structures (lanes 1, 2). Optimization of sorption and elution conditions by new structures differing in pore size, hydrophobic contribution, and pH allow the realization of IgG (IgG monoclonal antibody) purification.

FIG. 7 shows the chromatography of whey proteins on the large pore Mixed Mode resin (sorbent material). It is shown that IgG (e.g., IgG monoclonal antibodies) is isolated with other proteins (lanes 3, 4). For IgG purification, optimization of pH sorption-desorption conditions are needed.

FIG. 8 shows chromatography of whey proteins by large pore Mixed Mode resins (e.g., sorbent materials) at the initial pH of whey. It is shown from the demonstrated data that sorption at initial pH value of whey protein allows second step separation from low molecular mass (MM) proteins.

FIG. 9 shows the chromatography of whey proteins on the Mixed Mode resins (e.g., sorbent materials) with small pore size and considerable hydrophobic quality. Prepared data shows that new structures with small size of pores and required hydrophobic contribution allow separation of low MM proteins. Sorption conditions at pH 4 are the initial pH value of the whey proteins.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

When expressing amounts, ranges and sizes, dimensions and other measurable quantities the words “approximately” and “about” are used interchangeably.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A sorbent material comprising: a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, particles of particle sizes between approximately 10 micrometers to approximately 300 micrometers.

2. The sorbent material of claim 1, wherein the particle sizes are between approximately 100 micrometers to approximately 200 micrometers.

3. The sorbent material of claim 2, wherein the particles include pores of sizes of approximately 500 angstroms to approximately 3 micrometers.

4. The sorbent material of claim 3, wherein the pores are interconnected to each other.

5. The sorbent material of claim 1, wherein the volume of hydrophobic monovinyl monomers to hydrophilic monovinyl monomers is approximately 20:80.

6. The sorbent material of claim 1, wherein the matrix is bufferable to a pH of approximately 7.

7. The sorbent material of claim 1, wherein the monovinyl monomers include carboxyl groups from approximately 0.1 meq/ml to approximately 1.5 meq/ml.

8. The sorbent material of claim 7, wherein the carboxyl groups are approximately 0.7 meq/ml.

9. The sorbent material of claim 1, wherein the monovinyl monomers are selected from the group consisting of: monovinyl acids including acrylic acids and hydrophobic vinyl-containing compounds, including butylacrylate, tert-butylacrylate, butyl methacrylate, octyl and phenyl acrylate and methacrylates.

10. The sorbent material of claim 1, wherein the monovinyl monomers are selected from the group consisting of: vinyl contained compounds, including substituted amines, including ethyleneically substituted amines, allylamines, N-alkylethylenamine and dimethylaminoethyl methacrylate.

11. The sorbent material of claim 1, wherein the monovinyl monomers are selected from the group consisting of: hydrophobic vinyl contained compounds, including butylacrylate, tert-butylacrylate, butyl methacrylate, octyl and phenyl acrylate and methacrylates.

12. A method for isolation of Immunoglobulin G (IgG) from blood plasma comprising:
obtaining a sorbent material comprising:
a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers;
placing the sorbent material into a chromatographic column; and,
passing blood plasma through the chromatographic column to isolate the IgG.

13. The method of claim 12, additionally comprising: separating the isolated IgG of the blood plasma from the sorbent material.

14. The method of claim 13, wherein the separation of the IgG of the blood plasma from the sorbent material is performed by elution.

15. The method of claim 14, wherein the blood plasma is raw blood plasma.

16. The method of claim 15, wherein the raw blood plasma is mammalian blood plasma.

17. The method of claim 15, wherein the raw blood plasma is human blood plasma.

18. A method for isolation of Immunoglobulin G (IgG) monoclonal antibodies from whey of milk comprising:
obtaining a sorbent material comprising:
a plurality of cross linked monovinyl monomers defining a matrix, in a ratio of the volume of hydrophobic monovinyl monomers to hydrophilic monovinyl monomers of approximately 5:95 to approximately 40:60, the matrix being bufferable to pH ranges from approximately 5 to approximately 9, and, of particle sizes between approximately 10 micrometers to approximately 300 micrometers;
placing the sorbent material into a chromatographic column; and,
passing whey protein of the milk through the chromatographic column to isolate the IgG monoclonal antibodies.

19. The method of claim 18, wherein the milk includes transgenic milk.

20. The method of claim 19, wherein the transgenic milk is mammalian.
